(19) United States
(12) Patent Application Publication
(10) Pub. No.: US 2016/0030242 A1
(43) Pub. Date: Feb. 4, 2016

(54) Miniature Glaucoma Shunt

(71) Applicant: HANITA LENSES R.C.A. LTD., Hanita (IL)

(72) Inventors: Yakir KUSHLIN, Haifa (IL); Elie DAHAN, (US)

(21) Appl. No.: 14/776,519

(22) PCT Filed: Mar. 14, 2014
(86) PCT No.: PCT/US2014/026917

§ 371 Date: Sep. 14, 2015

Related U.S. Application Data
(60) Provisional application No. 61/781,752, filed on Mar. 14, 2013.

(51) Int. Cl. A61F 9/007

(52) U.S. Cl. CPC A61F 9/00781; A61F 2220/0008

(57) ABSTRACT

A glaucoma shunt (10) for reducing intraocular pressure (IOP) is provided. The glaucoma shunt (10, 50) includes a first planar element (12) from which extends a divider element (16). The first planar element (12) and the divider element (16) form two passageways (32) for aqueous evacuation.

### FIELD OF THE INVENTION

The present invention relates generally to an ophthalmic implant for treating glaucoma, and particularly to a miniature glaucoma shunt.

### BACKGROUND OF THE INVENTION

There are well known surgical techniques to treat glaucoma. The most widely used is the trabeculectomy, which involves removing a portion of scleral tissue that includes part of the trabecular meshwork and Schlemm's canal.

Another way to treat glaucoma is to surgically implant a drainage device in the eye. The drainage device functions to allow aqueous humor to drain from the anterior chamber and thereby reduce the intraocular pressure. The drainage device is usually implanted using an invasive surgical procedure. Pursuant to one such procedure, a flap is surgically cut in the sclera. The flap is folded back to form a small pocket and the drainage device is inserted into the eye through the flap. This procedure can be quite problematic as the implants are large and can result in various adverse events such as infections, erosions, and scarring, leading to the need to re-operate.

Another surgical technique, although currently less commonly performed, is the so-called Non-Penetrating Glaucoma Surgery (NPGS) in which the trabecular meshwork is left in place to act as a natural filtering membrane. NPGS includes variations such as Deep Sclerectomy, Viscocanalostomy and Canaloplasty. It is much safer than trabeculectomy, but requires extreme dexterity and a very long learning curve in order to be at least as efficient as trabeculectomy.

U.S. Pat. No. 7,118,547, to the co-inventor of the present invention, discloses a glaucoma surgery implant for non-penetrating glaucoma surgery (NPGS) made of a non-absorbable hydrophilic material. The implant is configured to be entirely covered by a scleral flap and totally contained in the intrascleral space.

U.S. Pat. No. 7,862,531, to the co-inventor of the present invention and a co-worker, provides a flow regulating implant with one or more grooves for allowing fluid flow.

### SUMMARY OF THE INVENTION

The present invention seeks to provide an improved miniature glaucoma shunt (MGS), as is described more in detail below.

The MGS of the present invention is made of biocompatible materials that have been widely used intraocularly for decades, such as but not limited to, poly(methyl methacrylate) (PMMA) and poly(2-hydroxyethyl methacrylate) (polyHEMA) or hydrophilic acrylic. The MGS of the present invention is minimally invasive, usually requiring an incision of less than 1 mm in length for its insertion. The MGS is placed under a scleral flap (similar to classic trabeculectomy) and does not require a high conjunctival bleb for its function. The aqueous from the anterior chamber is drained via the MGS into an intrascleral pocket (intrascleral bleb) and also suprachoroidally, and achieves a mainly blebless drainage of the aqueous humor.

The MGS of the present invention is advantageous over all the currently known drainage devices of the prior art used to treat glaucoma.

For example, tube shunts, such as the seton tube shunts (e.g., the Ahmed Valve, the Baerveldt and Molteno implants) are bulky and the surgery is heavily invasive. Consequently, setons are used primarily in end stage refractory glaucoma. In contrast, the MGS of the present invention is only a few millimeters in size and can be used in regular cases of glaucoma (open angle glaucoma), as well as in complicated cases (for example, narrow angle glaucoma, with the option of removing the natural lens of the eye beforehand, such as by phacoemulsification, in order to widen the angle of the eye). Setons are made from silicone tubes that are less biocompatible than the materials that can be used for the MGS of the present invention, such as PMMA, po1yHEMA, or other hydrophilic acrylic or hydrophobic acrylic materials. Setons have a considerable extrusion rate whereas it is unlikely that the MGS of the present invention placed under a scleral flap will extrude.

The EX-PRESS glaucoma filtration drainage device (from Alcon) is made of metal. Although the MGS of the present invention can be made of metal, it is preferably made of non-metallic biocompatible materials, as mentioned before, with which ophthalmologists are more familiar for surgical use in the eye. More importantly, the EX-PRESS needs an accurate direction for the pre-incision so the device tip does not touch the iris. The MGS of the present invention protrudes less into the anterior chamber than the EX-PRESS (1.5 mm versus 2.5 mm) and its tip is round and smooth and not sharp like the metallic EX-PRESS. The MGS of the present invention has at least two passageways for aqueous evacuation whereas the EX-PRESS has only one lumen, which can be blocked by blood or fibrin.

The iSTENT from Glaukos is a micro-invasive glaucoma surgery (MIGS) device that creates a permanent opening in the trabecular meshwork. The MGS of the present invention is much easier to insert than the iSTENT. The iSTENT is inserted into the Schlemm's canal using a surgical gonioscopy lens. This procedure is quite difficult because the patient' head has to be tilted to allow good visualization of the trabecular meshwork and requires a high skill technique from the surgeon. In contrast, the MGS of the present invention is easily inserted into the anterior chamber by direct visualization. The iSTENT is made of metal. Although the MGS of the present invention can be made of metal, it is preferably made of non-metallic biocompatible materials, as mentioned before. The same advantages hold for the MGS of the present invention versus the GOLD SHUNT from iMED PHARMA.

There is thus provided in accordance with an embodiment of the present invention a glaucoma shunt including a first planar element from which extends at least one divider element. The first planar element and the divider element form at least two passageways for aqueous evacuation. The divider preferably, but not necessarily, has a minimal height of 20 microns.

### BRIEF DESCRIPTION OF THE DRAWINGS

The present invention will be understood and appreciated more fully from the following detailed description, taken in conjunction with the drawing in which:

FIG. 1 is a simplified sectional illustration of a portion of the human eye; and

FIGS. 2-4 are simplified planar-view, sectional end-view and sectional side-view illustrations, respectively, of a glaucoma shunt, constructed and operative in accordance with an embodiment of the present invention, wherein FIG. 3 is taken along lines B-B in FIG. 2 and FIG. 4 is taken along lines A-A in FIG. 2; and

FIGS. 5-8 are simplified planar-view, end-view, side-view and perspective view illustrations, respectively, of a glaucoma shunt, constructed and operative in accordance with another embodiment of the present invention.

### DETAILED DESCRIPTION OF EMBODIMENTS

Reference is made to FIG. 1, which illustrates the anatomy of the human eye, and is presented here for better understanding of the implantation of the glaucoma shunt of the present invention.

The eye is covered on the outside by the sclera S and the cornea C. The conjunctiva CNJ lines the inside of the eyelids and covers the sclera S. The lens L is located near the front of the eye. The lens L provides adjustment of focus and is suspended within a capsular bag from the ciliary body CB, which contains the muscles that change the focal length of the lens. A volume in front of the lens L is divided into two by the iris I, which controls the aperture of the lens and the amount of light striking the retina. The pupil is a hole in the center of the iris I through which light passes. The volume between the iris I and the lens L is the posterior chamber PC. The volume between the iris I and the cornea C is the anterior chamber AC. Both chambers are filled with aqueous humor, a clear liquid. The posterior section of the eye is filled and supported by the vitreous body, a clear, jelly-like substance. The supraciliary space SCi is the region between the ciliary body CB and the sclera S, and the suprachoroidal space SCh is the region between the sclera S and the choroid Ch.

The ciliary body CB continuously forms aqueous humor in the posterior chamber PC by secretion from the blood vessels. The aqueous humor flows around the lens L and iris I into the anterior chamber AC and exits the eye through the trabecular meshwork, a sieve-like structure situated at the corner of the iris I and the wall of the eye (the iridocorneal angle). Some of the aqueous humor filters through the trabecular meshwork into Schlemm's canal, a small channel that drains into the ocular veins (aqueous veins). A smaller portion rejoins the venous circulation after passing through the ciliary body CB and eventually through the sclera S. This outflow path is known as the uveoscleral outflow path. In glaucoma one or more of the drainage flow paths becomes blocked.

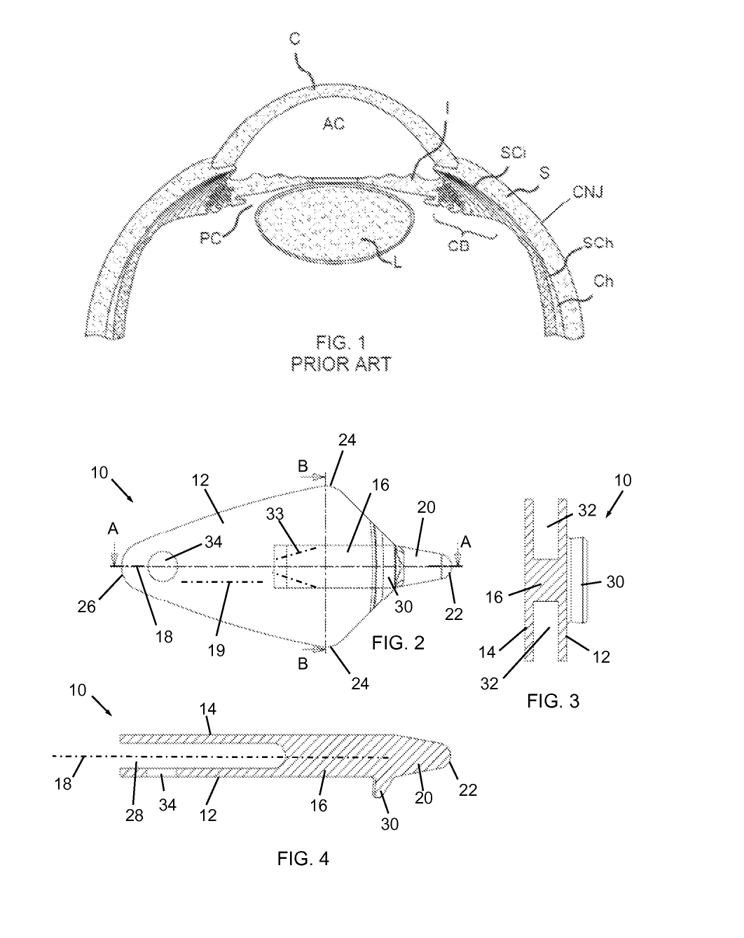

Reference is now made to FIGS. 2-4, which illustrate a glaucoma shunt 10, constructed and operative in accordance with a non-limiting embodiment of the present invention.

Glaucoma shunt 10 includes a first planar element 12 and a second planar element 14 that extend from opposite faces of a divider element 16, which may be positioned symmetrically on a central longitudinal axis 18 of elements 12 and 14. Alternatively, divider element 16 may be asymmetric with respect to the central longitudinal axis 18 of elements 12 and 14, as indicated by the broken line 19 in FIG. 2. A distal member 20 with a rounded tip 22 extends distally from a distal end of divider element 16 (or also from the distal ends of first and second planar elements 12 and 14). Distal member 20 has a streamlined or hydrodynamic shape (e.g., a rounded cone shape) for easy insertion of shunt 10 through the incision.

The first planar element 12 has a length and width extending along a planar surface 13 and a thickness perpendicular to the planar surface 13. The divider element 16 extends perpendicularly from planar surface 13. The divider element 16 is shorter than or equal to the length of first planar element 12 and the width of divider element 16 is less than the width of first planar element 12.

First and second planar elements 12 and 14 may gradually widen from their distal ends to a maximum width portion indicated by reference numeral 24 (FIG. 2) and then taper to a rounded proximal extremity 26. Shapes of first and second planar elements 12 and 14 may include, without limitation, egg and leaf shapes, e.g., ovate, obovate, cuneate, truncate and others. In one embodiment, first and second planar elements 12 and 14 are substantially parallel to each other. First and second planar elements 12 and 14 can be flexible to bend inwards or outwards with respect to each other.

The portions of first and second planar elements 12 and 14 proximal to divider element 16 are separated by a gap 28. First and second planar elements 12 and 14, divider element 16 and gap 28 form two flow passageways 32 (FIG. 3) for aqueous evacuation. The proximal end of divider element 16 may have a streamlined or hydrodynamic shape for reducing drag of the liquid flow through passageways 32, as indicated by broken lines 33 in FIG. 2.

A stopper element 30, which may be shaped like a dorsal fin, extends from a distal portion of one of first and second planar elements 12 and 14, such as first planar element 12. Stopper element 30 may be useful in preventing egress (extrusion) of the shunt 10 from the eye after implantation. Stopper element 30 can easily be inserted through the small insertion incision at an angle.

The MGS 10 is only a few millimeters in size (from distal to proximal ends the length can be 3-6 mm, with a maximum width portion 24 of about 2 mm and plate thickness of 0.1-0.3 mm); the invention is not limited to these dimensions. The MGS 10 can be placed under a scleral flap and is small in size so it does not have the disadvantages of trying to insert large devices in a scleral flap. The MGS 10 protrudes only about 1.5 mm into the anterior chamber; the invention is not limited to these dimensions. The MGS 10 is easily inserted into the anterior chamber by direct visualization.

In one embodiment, both first and second planar elements 12 and 14 are inserted into the intrascleral space, that is, both are situated in the sclera S. In another embodiment, one of the first and second planar elements 12 and 14 is inserted into the suprachoroidal space SCh while the other one is inserted into the intrascleral space.

One of first and second planar elements 12 and 14 may be formed with an opening 34, which may assist in fixation of MGS 10. For example, a suture (not shown) may be passed through opening 34 for tying to tissue. In other embodiment, when the element with opening 34 is inserted into the suprachoroidal space SCh, choroidal tissue may jut through opening 34 and adhere to the sclera S, without any need for a suture.

Accordingly, shunt 10 drains aqueous humor from the anterior chamber of the eye into the subscleral, suprachoroidal and subconjunctival spaces, thereby reducing intraocular pressure (TOP). Shunt 10 achieves a mainly blebless drainage of the aqueous humor.

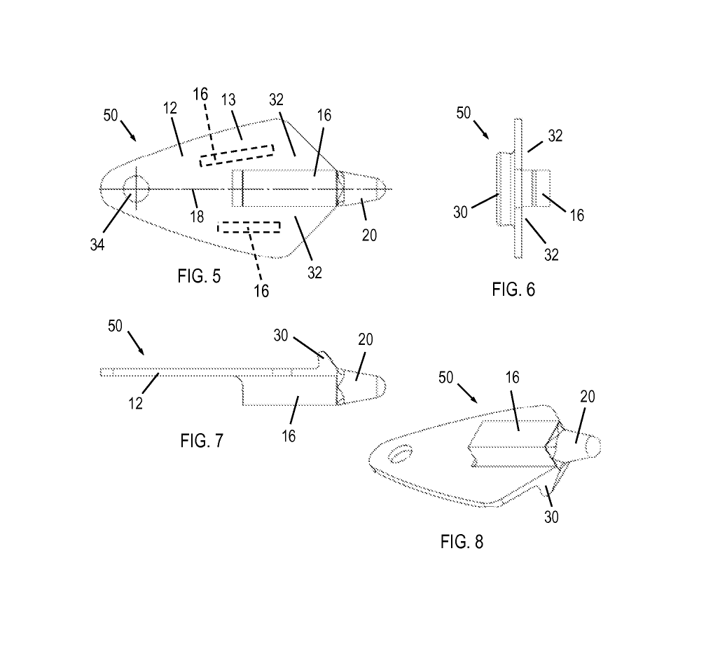

Reference is now made to FIGS. 5-8, which illustrate a glaucoma shunt 50, constructed and operative in accordance with a non-limiting embodiment of the present invention.

Glaucoma shunt 50 is constructed basically the same as shunt 10, except that shunt 50 has only the first planar element 12 and does not have a second planar element. As with shunt 10, in shunt 50 the divider element 16 creates two flow passageways 32 for aqueous evacuation, except that the passageways are not bound by a second planar element. In all embodiments of the invention, more dividers may be added to the central divider in order to optimize the flow of the aqueous humor through the implant. The additional dividers may be parallel or not parallel to the first divider (as shown in broken lines in FIG. 5), and as mentioned previously, the first divider does not have to be central.

1.-13. (canceled)

14. A glaucoma shunt comprising:
two flexible, substantially parallel planar elements;
a divider disposed between said two planar elements, wherein:
(a) said divider has a width smaller than widths of said two planar elements, thereby defining two flow passageways on two sides of said divider, each of said two flow passageways delimited by said divider and portions of said two planar elements,
(b) said divider has a length smaller than lengths of said two planar elements, such that said divider ends proximally before said two planar elements end; and
a distal member extending distally from a distal end of said divider and distal ends of said two planar elements.

15. The glaucoma shunt according to claim 14, further comprising a stopper extending from a distal portion of one of said two planar surfaces.

16. The glaucoma shunt according to claim 15, wherein said stopper element is shaped as a dorsal fin oriented proximally.

17. The glaucoma shunt according to claim 14, having a width of up to about 2 millimeters.

18. The glaucoma shunt according to claim 14, having a length of between 3 and 6 millimeters from a distal to a proximal end thereof.

19. The glaucoma shunt according to claim 14, wherein each of said two planar surfaces gradually widens from its distal end to a maximum width portion, and then tapers to a proximal extremity.

20. The glaucoma shunt according to claim 19, wherein said proximal extremity of each of said two planar surfaces is rounded.

21. The glaucoma shunt according to claim 14, wherein one of said two planar elements has a proximally-located opening.

22. The glaucoma shunt according to claim 14, wherein each of said two planar elements has a thickness of between 0.1 and 0.3 millimeters.

23. The glaucoma shunt according to claim 14, wherein said distal member has a distal rounded tip.

24. The glaucoma shunt according to claim 14, wherein said distal member has a streamlined shape.

25. The glaucoma shunt according to claim 24, wherein said distal member has a rounded cone shape.

26. The glaucoma shunt according to claim 14, made of a material selected from the group consisting of:
poly(methyl methacrylate) (PMMA),
poly(2-hydroxyethyl methacrylate) (polyHEMA), and
hydrophilic acrylic.

27. A method for implanting a glaucoma shunt, the method comprising:
forming a scleral flap;
inserting the glaucoma shunt under the scleral flap, wherein the glaucoma shunt comprises:
(i) two flexible, substantially parallel planar elements,
(ii) a divider disposed between said two planar elements, wherein:
(a) said divider has a width smaller than widths of said two planar elements, thereby defining two flow passageways on two sides of said divider, each of said two flow passageways delimited by said divider and portions of said two planar elements,
(b) said divider has a length smaller than lengths of said two planar elements, such that said divider ends proximally before said two planar elements end,
(iii) a distal member extending distally from a distal end of said divider and distal ends of said two planar elements.

28. The method according to claim 27, wherein the inserting comprises making the glaucoma shunt protrude about 1.5 millimeters into the anterior chamber of the eye.

29. The method according to claim 27, wherein the forming of the scleral flap comprises making an incision of 1 millimeter or less in length.
